(19) United States
(12) Patent Application Publication
(10) Pub. No.: US 2009/0259066 A1
(43) Pub. Date: Oct. 15, 2009

(54) Method for preparing prostaglandin F analogue

(75) Inventors: Chi-Hsiang Yao, Taoyuan Hsien (TW); Chun-Mei Yang, Taoyuan Hsien (TW); Hou-Hsun Chao, Taoyuan Hsien (TW); Ge-Ray Lee, Taoyuan Hsien (TW)

Correspondence Address: BACON & THOMAS, PLLC, 625 SLATERS LANE, FOURTH FLOOR, ALEXANDRIA, VA, 22314-1176, US

(73) Assignee: Everlight USA, Inc., Pineville, NC (US)

(21) Appl. No.: 12/216,384

(22) Filed: Jul. 3, 2008

(30) Foreign Application Priority Data
Apr. 9, 2008 (CN) 200810091707.2

(51) Int. Cl. C07C 67/00; C07C 231/00

(52) U.S. Cl. 560/60; 564/134

(57) ABSTRACT

A method for preparing a prostaglandin F analogue represented by the following formula (I) is disclosed,

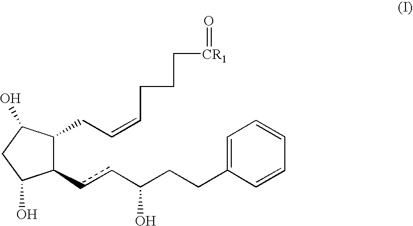

wherein R₁, and [symbol] are as defined in the specification.

### BACKGROUND OF THE INVENTION

1. Field of the Invention

The present invention relates to a method for preparing prostaglandin F analogue.

2. Description of Related Art

The derivatives of prostaglandin F-type are capable using in clinical treatments for glaucoma or high intraocular pressure caused by other reasons. Glaucoma is a disease caused by a continued or intermittent high intraocular pressure. The continued high pressure will bring damage to eyeball tissues and vision ability, if it didn't serve treatment in time, it will also bring damage to optic nerve system and lead to vision recession or narrowed eyesight. The most serious situation is losing vision. Presently, glaucoma is one of the three critical diseases causing blind. F-type show good curative effect in treating glaucoma and high intraocular pressure that caused by other reasons. Therefore, the using and preparing method of prostaglandin F-type compound now become a main focus for many chemists and pharmacists. Some prior arts have disclosed methods for preparing and using the derivatives of prostaglandin, such as those disclosed in U.S. Pat. No. 4,599,353, Europe patent No. 364,417, No.495069, No. 544,899, PCT publication No. WO95/11003, WO01/055101, WO01/087816, WO02/096868, WO02/096898, and WO03/008368.

### SUMMARY OF THE INVENTION

The present invention relates to a method for preparing prostaglandin F analogue.

The present invention provides a method for preparing prostaglandin F analogue represented by the following formula (I):

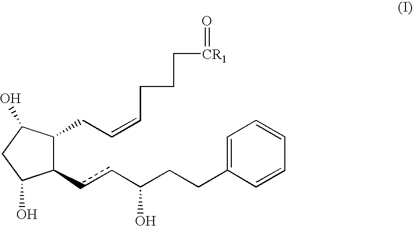

wherein R₁ is C_{1˜6} alkoxy, or C_{1˜6} alkylamino, and [symbol] is a single bond or a double bound.

The method for preparing the compound related to the formula (I) of the present invention comprises the following steps:

(a) reacting a compound of the following formula (II) in the presence of (−)-chlorodiisopinocamphenylborane,

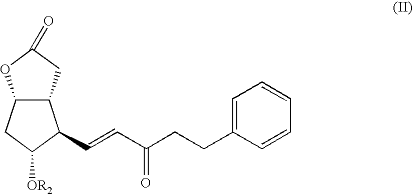

wherein
R₂ is tetrahydropyranyl substituted or unsubstituted with C_{1˜6} alkyl, or a protecting group as shown below:

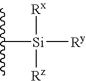

wherein R^{x}, R^{y}, or R^{z} are the same or different, and each independently C_{1˜6} alkyl, C_{6˜10} aryl, or C_{7˜16} arylalkyl,

and performing stereoselective reduction to form a compound of the following formula (III):

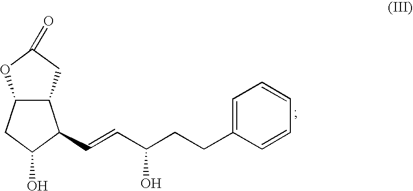

(b) performing protection reaction of the compound of formula (III) to form a compound of the following formula (IV),

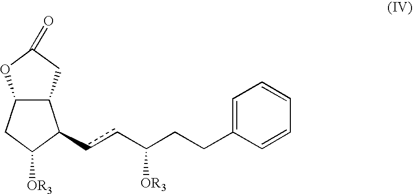

wherein
R₃ is tetrahydropyranyl substituted or unsubstituted with C_{1˜6} alkyl, or a protecting group as shown below:

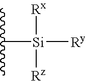

wherein R^{x}, R^{y}, or R^{z} are the same or different, and each independently C_{1˜6} alkyl, C_{6˜10} aryl, or C_{7˜16} arylalkyl;

(c) reducing the compound of the formula (IV) to form a compound of the following formula (V):

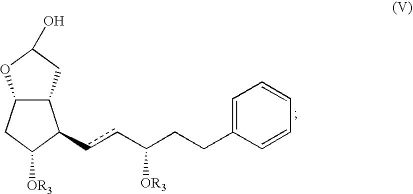

(d) performing Witting reaction of the compound of the following formula (V) with a compound represented as follow,

HOOC(CH₂)₄P⁺(R^{a})₃Y⁻

wherein R^{a} is C_{1˜6} alkyl, or C_{6˜10} aryl; Y is F, Cl, Br, or I,

to form a compound of the following formula (VI):

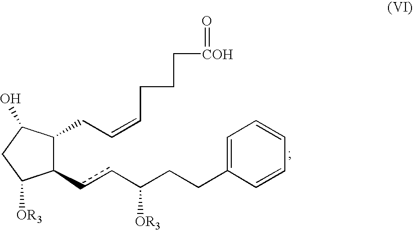

(e) performing esterification of the compound of the formula (VI) with a compound represented as follow,

R₄-Z

wherein R₄ is H, or C_{1˜6} alkyl, and Z is halogen, sulphate, mesyl, tosyl, or C_{1˜6} hydroxyl,

to form a compound of the following formula (VII):

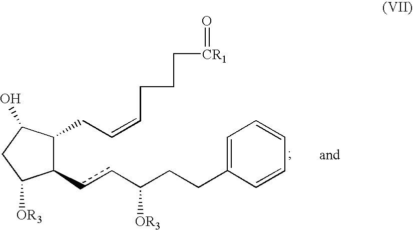

(f) deprotecting the compound of the formula (VII) to form a prostaglandin F analogue represented by the formula (I).

Furthermore, the present invention also provides another method for preparing prostaglandin F analogue represented by the formula (I), which comprises the following steps:

(a) reacting a compound of the following formula (II) in the presence of (−)-chlorodiisopinocamphenylborane,

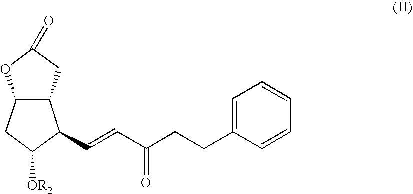

wherein
R₂ is tetrahydropyranyl substituted or unsubstituted with C_{1˜6} alkyl, or a protecting group as shown below:

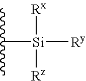

wherein R^{x}, R^{y}, or R^{z} are the same or different, and each independently C_{1˜6} alkyl, C_{6˜10} aryl, or C_{7˜16} arylalkyl,

and performing stereoselective reduction to form a compound of the following formula (III):

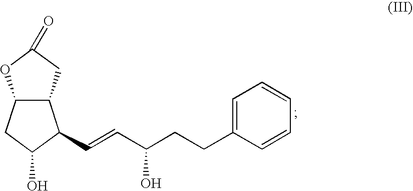

(b) performing protection reaction of the compound of formula (III) to form a compound of the following formula (IV),

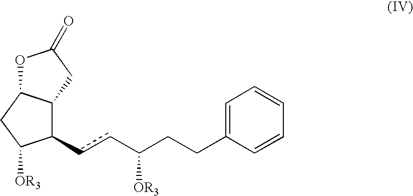

wherein
R₃ is tetrahydropyranyl substituted or unsubstituted with C_{1˜6} alkyl, or a protecting group as shown below:

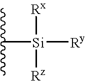

wherein R^{x}, R^{y}, or R^{z} are the same or different, and each independently C_{1˜6} alkyl, C_{6˜10} aryl, or C_{7˜16} arylalkyl;

(c) reducing the compound of the formula (IV) to form a compound of the following formula (V):

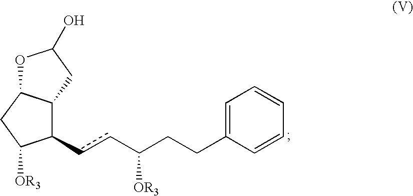

(d) performing Witting reaction of the compound of the following formula (V) with a compound represented as follow,

HOOC(CH₂)₄P⁺(R^{a})₃Y⁻

wherein R^{a} is C_{1˜6} alkyl, or C_{6˜10} aryl; Y is F, Cl, Br, or I,

to form a compound of the following formula (VI):

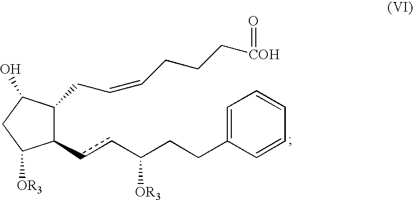

(e)′ deprotecting the compound of the formula (VI) to form a compound of the following formula (VIII):

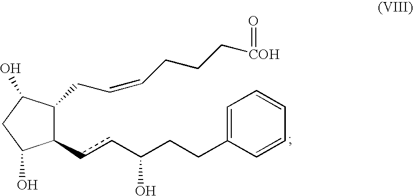

(f)′ performing esterification of the compound of the formula (VIII) to form a compound of the following formula (IX):

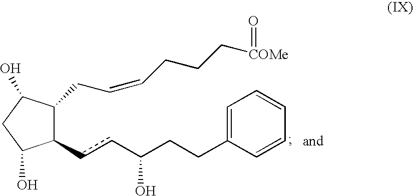

(g) performing amidation of the compound of the formula (IX) with a compound represented as follow,

R₅—NH₂

wherein R₅ is C_{1˜6} alkyl,

to form a prostaglandin F analogue represented by the formula (I).

In the two above-mentioned methods of the present invention, the step (b) comprises step (b1) and step (b2):

(b1) hydrogenating the compound of the formula (III) to form a compound of the following formula (III)′:

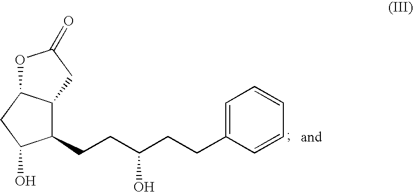

(b2) protecting the compound of formula (III)′ to form the compound of the following formula (IV),

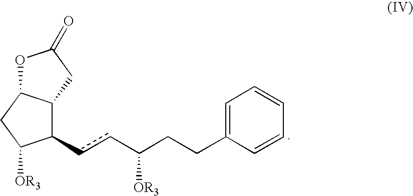

In addition, in the compound of the formula (I), R₁ may be isopropoxy, or ethylamino.

In the compound of the formula (II), R₂ may be tetrahydropyranyl, triethylsilyl, tert-butyldimethylsilyl, t-butyldiphenylsilyl, or dimethylphenylsilyl.

In the compounds of the formula (V) and the formula (VI), R₃ may be tetrahydropyranyl, triethylsilyl, tert-butyldimethylsilyl, t-butyldiphenylsilyl, or dimethylphenylsilyl.

Furthermore, in one aspect of the present invention, the compound of the formula (I) may be

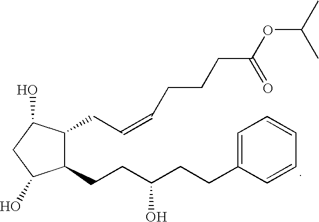

In another aspect of the present invention, the compound of the formula (I) may be

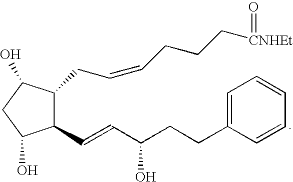

Other objects, advantages, and novel features of the invention will become more apparent from the following detailed description.

### DETAILED DESCRIPTION OF THE PREFERRED EMBODIMENT

The present invention relates to a method for preparing the prostaglandin F analogue represented by a compound of the following formula (I):

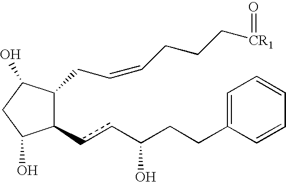

wherein R₁ is C_{1˜6} alkoxy, or C_{1˜6} alkylamino, and [symbol] is a single bond or a double bound.

Particularly, in the method for preparing the compound of the formula (I), the step comprises: reacting a compound of the following formula (II) in the presence of (−)-chlorodiisopinocamphenylborane, and performing stereoselective reduction,

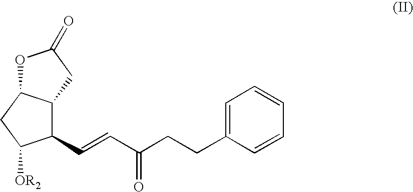

wherein
R₂ is tetrahydropyranyl substituted or unsubstituted with C_{1˜6} alkyl, or a protecting group as shown below:

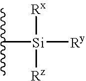

wherein R^{x}, R^{y}, or R^{z} are the same or different, and each independently C_{1˜6} alkyl, C_{6˜10} aryl, or C_{7˜16} arylalkyl.

After the stereoselective reduction of the compound of the formula (II), a compound of the following formula (III) with excellent enantiomeric purity is formed,

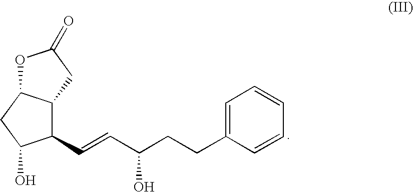

The compound of the formula (III) of the present invention, which has excellent enantiomeric purity, can be used for manufacturing a prostaglandin F analogue represented by the formula (I) having good quality.

A preferred process for synthesis of the compound of the formula (I) is shown as the following scheme 1.

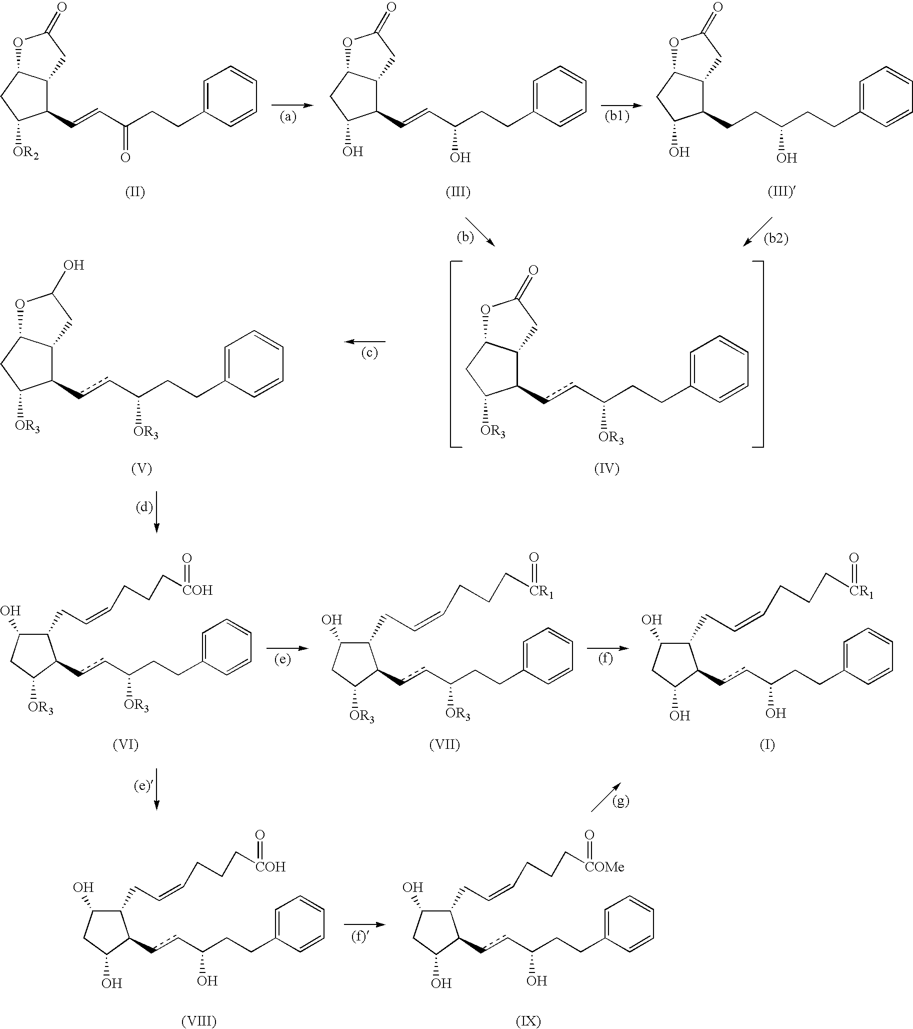

The definitions of R₁, R₂, and [symbol] are the same as described before. R₃ is tetrahydropyranyl substituted or unsubstituted with C_{1˜6} alkyl, or a protecting group as shown below:

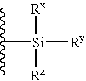

wherein R^{x}, R^{y}, or R^{z} are the same or different, and each independently C_{1˜6} alkyl, C_{6˜10} aryl, or C_{7˜16} arylalkyl.

Hereafter, examples are provided for illustrating the steps of the method for preparing the prostaglandin F analogue represented by the formula (I) of the present invention. As shown in scheme 1, the methods for preparing the prostaglandin F analogue of the present invention comprise following steps:
(a) reducing the compound of the formula (II) to form the compound of the formula (III):

The compound of the formula (II) is added into an organic solvent, and then (−)-chlorodiisopinocamphenylborane is added therein to form a mixture. The temperature of the mixture is maintained around −75° C. to 0° C. At the same time, the 15-position of the compound of the formula (II) is stereoselectively reduced to form a 15-S enantiomer as the main product. After the product is warm to the room temperature (about 25° C.), a compound of the formula (III) is obtained.

In the aforementioned step (a), the organic solvent used herein may be the conventional aprotic solvent, such as tetrahydrofuran (THF), dimethylformamide (DMF), dimethyl sulfoxide (DMSO), toluene, ether, dichloromethane, or dichloroethane. Most preferably, the organic solvent is THF.
(b) protecting the compound of the formula (III) to form the compound of the formula (IV):

The compound of the formula (III) is reacted with dihydropyran substituted or unsubstituted with C_{1˜6} alkyl, or reacted with a Silylation agent as shown below, to form the compound of the formula (V).

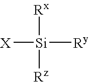

R^{x}, R^{y}, or R^{z} are the same or different, and each independently C_{1˜6} alkyl, C_{6˜10} aryl, or C_{7˜16} arylalkyl. X is F, Cl, Br, or I.

The organic solvent used herein may be any conventional aprotic solvent, such as THF, DMF, DMSO, toluene, ether, dichloromethane, or dichloroethane. Most preferably, the organic solvent is medium to high polar solvent, such as THF, DMF, toluene, or ether.

In addition, the example of dihydropyran substituted or unsubstituted with C_{1˜6} alkyl is 3,4-dihydro-2H-pyran. The example of Silylation agent is triethylsilyl chloride, tert-butyldimethylsilyl chloride, tert-butyldiphenylsilyl chloride, or dimethylphenylsilyl chloride. More preferably, triethylsilyl chloride is used herein.

Furthermore, the reaction temperature in the step (b) may be between 30 to −10° C. Preferably, the reaction temperature in the step (b) is between 0 to 5° C.

Moreover, after the step (a), it is possible to perform step (b1) followed by step (b2) to form the compound of the formula (IV).
(b1) hydrogenating the compound of the formula (III) selectively if needed, to form the compound of the following formula (III)′:

The compound of the formula (III) is dissolved in an organic solvent, and then metal catalyst and hydrogen are added therein to perform hydrogenation. Examples of the organic solvent used herein may be methanol, ethanol, isopropanol, ethyl acetate, THF, or toluene. Preferably, the organic solvent is methanol, THF, or ethyl acetate. More preferably, the organic solvent is methanol.

Besides, examples of the metal catalyst used in the hydrogenation of the step (b1) may be Raney Ni, Rh, Ru, Ir, Pt, or Pd. Preferably, the metal catalyst is Pt, or Pd. Most preferably, the metal catalyst is Pd.

In the present invention, the reaction temperature for the hydrogenation may be between 0° C. to 40° C., which depends on the organic solvent used in the hydrogenation. In addition, the reaction time of the hydrogenation is unlimited. Preferably, the reaction time is 1 to 2 hours.
(b2) protecting the compound of the formula (III)′ to form the compound of the following formula (IV):

The same reaction condition described in step (b) are used herein to perform a protection reaction of the compound of the formula (III)′. Then, the compound of the formula (IV) is obtained.
(c) reducing the compound of the formula (IV) to form the compound of the following formula (V):

The compound of the formula (IV) is dissolved in an organic solvent, and a reduction agent is added therein under low temperature to perform reduction reaction. The lactone group of the compound of the formula (IV) is reduced to a lactol group, and then the compound of the formula (V) is obtained.

In the reduction reaction of step (c), the organic solvent can be any conventional aprotic solvent, such as THF, toluene, ether, dichloromethane, or dichloroethane. Preferably, the organic solvent is THF, toluene, or ether. Most preferably, the solvent is THF, or toluene.

Example of the reduction agent used herein may be diisobutylaluminium hydride (DIBAL-H).

In addition, the temperature of the reduction reaction of the step (c) may be between −60° C. to −80° C. More preferably, the temperature is between −60° C. to −70° C.
(d) performing Witting reaction of the compound of the formula (V) to form the compound of the formula (VI):

The compound of the formula (V) and a compound represented as follow are dissolved in an organic solvent;

HOOC(CH₂)₄P⁺(R^{a})₃Y⁻

wherein R^{a} is C_{1˜6} alkyl, or C_{6˜10} aryl; and Y is F, Cl, Br, or I.

After an alkaline agent is added therein, Witting reaction is performed. Then, the compound of the formula (VI) is obtained.

In Witting reaction, the organic solvent can be conventional high polar solvent, medium polar solvent, or chlorinated solvent, such as THF, toluene, dichloromethane, dichloroethane or ester. Preferably, the organic solvent is THF, or toluene. Most preferably, the organic solvent is THF.

The alkaline agent may be conventional organic alkaline, or inorganic alkaline, such as triethylamine, diisopropylethylamine, DBU, sodium hydride (NaH), potassium carbonate, or potassium tert-butoxide. Preferably, the alkaline agent is potassium tert-butoxide, triethylamine, potassium carbonate, or NaH. Most preferably, the alkaline agent is tert-butoxide

In addition, the temperature for performing Witting reaction in the step (d) may be between −20° C. to 40° C. Preferably, the temperature is between 0° C. to 5° C.
(e) performing esterification of the compound of the formula (VI) to form the compound of the formula (VII):

The compound of the formula (VI) and a compound represented as follow are dissolved in an organic solvent;

R₄-Z

wherein R₄ is H or C_{1˜6} alkyl; and Z is halogen, sulphate, mesyl, tosyl, or C_{1˜6} hydroxyl.

After an acidic or an alkali catalyst is added therein, esterification is performed. Then, the compound of the formula (VII) is obtained.

The organic solvent used herein may be a conventional polar solvent, such as THF, DMF, DMSO, toluene, ether, dichloromethane, dichloroethane, methanol, ethanol, isopropanol, or acetone. Preferably, the organic solvent is THF, DMF, alcohol, or acetone.

In addition, the catalyst used for esterification may be conventional organic acids or organic bases, such as triethylamine, diisopropylethylamine, pyridinium p-toluenesulfonate (PPTS), p-Toluene sulfonic acid (PTSA), or DBU. Preferably, the catalyst is triethylamine, diisopropylethylamine, or DBU.

Furthermore, the temperature for performing esterification may be between 40 to −10° C. More preferably, the temperature is between 20 to 25° C.
(f) deprotecting the compound of the formula (VII) to form a prostaglandin F analogue represented by the formula (I):

The compound of the formula (VII) is dissolved in an organic solvent, an aqueous solution, or a mixture of solvents and aqueous solutions with various ratios. After an acidic catalyst is added therein, a protection reaction is performed. Then, the compound represent by the formula (I) is obtained.

The solvent used herein may be a conventional polar solvent, such as THF, methanol, ethanol, isopropanol, or acetone. Preferably, the organic solvent is THF, alcohol, or acetone.

In addition, the catalyst used herein may be conventional inorganic acids and organic acids, such as PPTS, PTSA, hydrochloric acid (HCl), or acetic acid. Preferably, the catalyst is PPTS, PTSA, or HCl.

Furthermore, the temperature for performing deprotection reaction may be between 50 to −10° C. Preferably, the temperature is between 0 to 5° C.
(e)′ deprotecting the compound of the formula (VI) to form the compound of the formula (VIII):

The same reaction condition and the same catalyst described in step (f) are used herein to perform a protection reaction of the compound of the formula (VI). Then, the compound of the formula (VIII) is obtained.
(f)′ performing esterification of the compound of the formula (VIII) to form the compound of the following formula (IX):

The compound of the formula (VIII) and methyl iodide is dissolved in an organic solvent. After an acidic or an alkali catalyst is added therein, esterification is performed. Then, the compound of the formula (IX) is obtained.

The organic solvent used herein may be a conventional polar solvent, such as THF, DMF, DMSO, toluene, ether, dichloromethane, dichloroethane, methanol, ethanol, isopropanol, or acetone. Preferably, the organic solvent is THF, DMF, alcohol, or acetone.

In addition, the catalyst used for esterification may be conventional organic acids or organic bases, such as triethylamine, diisopropylethylamine, PPTS, PTSA, or DBU. Preferably, the catalyst is triethylamine, diisopropylethylamine, or DBU.

Furthermore, the temperature for performing esterification may be between 40 to −10° C. More preferably, the temperature is between 20 to 25° C.
(g) performing amidation of the compound of the formula (IX) to form a prostaglandin F analogue represented by the formula (I):

The compound of the formula (IX) and an alkylamine represented as follow,

R₅—NH₂

wherein R₅ is C_{1˜6} alkyl,

are dissolved in an organic solvent, an aqueous solution, or a mixture of solvents and aqueous solutions with various ratios. After the amidation is performed, the compound of the formula (I) is obtained.

The organic solvent used herein may be any conventional aprotic solvent, such as THF, DMF, DMSO, toluene, ether, dichloromethane, or dichloroethane. More preferably, the organic solvent is medium to high polar solvent, such as THF, DMF, toluene, or ether.

On the other hand, the compound of the formula (II) of the present invention can be obtained by reacting commercial Protected-Corey aldehyde with a phosphonate compound through Wittig reaction, wherein the example of the phosphonate compound may be dimethyl-2-oxo-4-phenylbutylphosphonate. Other detail description will be illustrated in the following examples.

In addition, the advantage of the present invention is that, the 15-position of the compound of the formula (II) having the acidic protecting group is stereoselectively reduced under the presence of (−)-chlorodiisopinocamphenylborane to form a 15-S enantiomer as the main product, and the deprotection reaction is performed at the same time to obtain the compound of the formula (III). For example, in the example 2, the ratio of 15-S enantiomer to 15-R enantiomer is 95.6:4.4. A lot of patents, such as WO03/008368 and WO02/096898, have disclosed that the starting material having the basic protecting group is stereoselectively reduced under the presence of (−)-chlorodiisopinocamphenylborane, and then the deprotection reaction is performed under basic condition. However, in the present invention, the compound of the formula (II) having acidic protecting group is reduced under the presence of (−)-chlorodiisopinocamphenylborane. Furthermore, the deprotection reaction can be performed at the same time under the same condition as the reduction reaction of the compound of the formula (II). Hence, the diastereomeric excess (de) value of the product is excellent in the present invention.

Furthermore, the acidic protection group described herein is a functional group, which can be deprotected to a hydroxyl group under an acidic condition. The basic protection group described herein is a functional group, which can be deprotected to a hydroxyl group under a basic condition.

The diastereomeric excess (de) value illustrated herein means that, the value of (15-S enantiomer−15-R enantiomer)/(15-S enantiomer+15-R enantiomer) can achieve 90% or more. Additionally, some priors have disclosed that it is possible to use different reducing agent. For example, WO02/096868 has disclosed that the reducing system of (R)-Methyl Oxazaborolidine (MeCBS)/Dimethylsulfide Borane (DMSB) can be used for performing reduction reaction. Besides, the reducing system of MeCBS/Borane-Tetrahydrofuran Complex (BTHF) or the reducing system of MeCBS/BH3-NR3 can be used for performing reduction reaction, wherein the example of BH3-NR3 is N,N-diethylanilineborane (DEANB). However, the de value cannot achieve 90% or more by using these reducing systems, but the de value of the product prepared by the method of the present invention can achieve 90% or more. Hence, the method of the present invention is better than the method illustrated in the prior.

The present invention provides the following examples for illustration in detail.

Although the present invention has been explained in relation to its preferred examples, it is to be understood that many other possible modifications and variations can be made without departing from the scope of the invention as hereinafter claimed. Without specific explanations, the unit of the percentages used in the examples is calculated by weight, and the temperature is represented by Celsius degrees (° C.).

### EXAMPLE 1

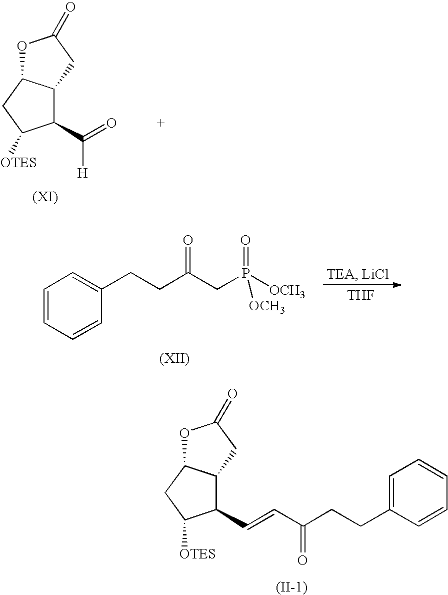

6.71 g of LiCl and 30 ml of THF were added into a 250 ml three-necked flask, and then the compound of the formula (XII)/THF solution (9.01 g of the compound of the formula (XII) in 40 ml of THF) was added into the mixture. The TEA/THF solution (9.88 ml of TEA in 20 ml of THF) was added into the mixture dropwise under −15˜−5° C. After the mixture was stirred for 30 min, the compound of the formula (XI)/THF solution (10 g of the compound of the formula (XI) in 40 ml of THF) was added into the mixture. After reaction for 1 hour, TLC was used to ensure the completion of the reaction. When the reaction was completed, 100 ml of water was added into the mixture to stop the reaction under room temperature. Then, 100 ml of ethyl acetate is used for extraction twice. The upper layer was separated, and sodium sulfate was added into the upper layer for dehydration. After the sodium sulfate was filtered out, the filtrate was concentrated under reduced pressure. Then, the concentrated filtrate was applied into column chromatography, and eluted by the eluent (ethyl acetate/hexane=1/4). Finally, 7.44 g of an oil-like product (II-1) was obtained, and the yield of the product (II-1) was 51%.

¹H NMR (CDCl₃):

δ: 7.17-7.31 (m, 5H), 6.52-6.60 (dd, 1H), 6.11-6.17 (d, 1H), 4.93-4.94(q, 1H), 4.0-4.02 (q, 1H), 2.29-2.96 (m, 9H), 1.95-2.05 (m, 1H), 0.89-0.94 (t, 9H), 0.51-0.59 (q, 6H)

### EXAMPLE 2

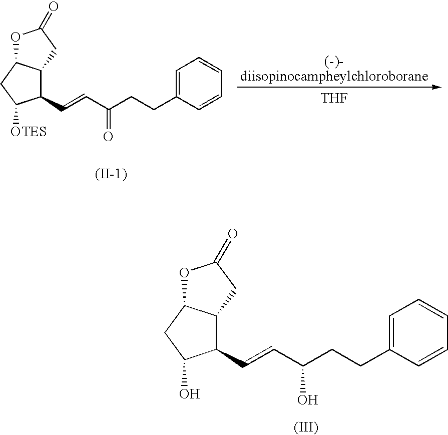

12.05 g of the compound of the formula (II-1) and 120 ml of THF were added into a 500 ml three-necked flask, and then 54.26 g of (−)-chlorodiisopinocamphenylborane was added into the mixture dropwise under −60° C.˜−75° C. The mixture was warm to room temperature and stirred for 15 hours. After the reaction was completed, 80 ml of saturated NaHCO_{3(aq)} was added into the mixture, and the pH of the mixture was adjusted to 6˜7. Then, 80 ml of ethyl acetate was added into the mixture to extract the mixture three times. The upper layer was separated, and 100 ml of water was added therein to wash the upper layer. Sodium sulfate was added into the upper layer for dehydration. After sodium sulfate was filtered out, the filtrate was concentrated under reduced pressure. Then, 47.3 g of an oil-like crude product was obtained. The crude product was extracted by 180 ml Acetonitrile and 85 ml Hexane. After the layers were formed, the lower layer was separated and 85 ml of hexane was added to extract the lower layer three times. After extraction, the lower layer was separated and concentrated under reduced pressure to obtain 11.57 g of an oil-like product (III). The chemical purity of the oil-like product (III) is 87.26%, and the de value of which is 91.2%. Finally, the oil-like product (III) was applied into column chromatography, and eluted by the eluent (ethyl acetate/hexane=1/1 and 1/2) to get 6.9 g of the oil-like product (III). The chemical purity of the oil-like product (III) is 98.22%.

The de value of the compounds was analyzed by HPLC. The analysis condition for HPLC was set as follow:

1. Column: Rx sil 4.6*250mm

2. Mobile phase: hexane/ethanol=9/1

3. Flow rate: 1.5 ml/min

4. Temperature: 30° C.

5. Retention time: 13.86 min for 15-S enantiomer
15.12 min for 15-R enantiomer

### EXAMPLE 3

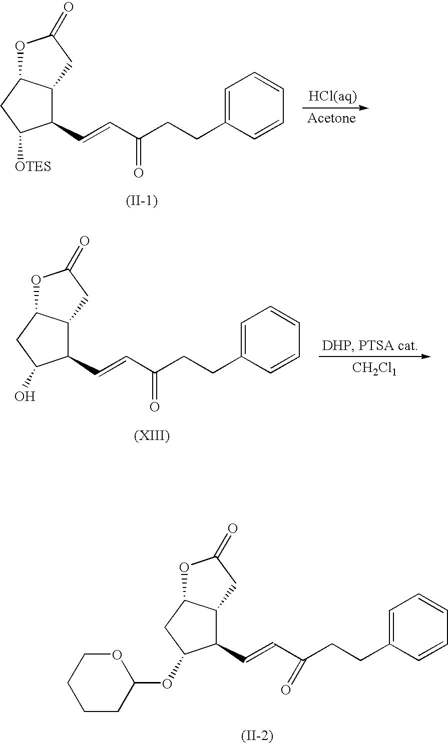

1.13 g of the compound of the formula (II-1) and 11 ml of acetone were added into a 50 ml flask, and the 19% of HCl(aq) was used to adjust the pH of the mixture less than 2. After the mixture was stirred for 30 min, TLC was used to ensure the completion of the reaction. When the reaction was completed, saturated NaHCO_{3(aq)} was added into the mixture, and the pH of the mixture was adjusted to 6˜7. After the mixture was concentrated under reduced pressure, 10 ml of ethyl acetate was used for extraction three times. After the layers were formed, the upper layer was separated, and sodium sulfate was added into the upper layer for dehydration. After sodium sulfate was filtered out, the filtrate was concentrated under reduced pressure to get 1.04 g of an oil-like crude product (XIII). Sequentially, the crude product (XIII) was used for the next process directly.

1.04 g of the crude product (XIII), and 10 ml of dichloromethane (CH₂Cl₂) and PTSA as catalyst, were added into 100 ml flask. Then, 0.47 ml of DHP was added into the mixture, and the mixture was stirred for 1 hour. TLC was used to ensure the completion of the reaction. When the reaction was completed, 10 ml of water was used for extraction three times, and the upper layer was collected. Then, sodium sulfate was added into the upper layer for dehydration. After sodium sulfate was filtered out, the filtrate was concentrated under reduced pressure to get 1.25 g of an oil-like crude product. The oil-like crude product was applied into column chromatography, and eluted by the eluent (ethyl acetate/hexane=1/3) to get 0.79 g of an oil-like product (II-2).

¹H NMR (CDCl₃):
δ: 7.30-7.15 (m, 5H), 6.64-6.54 (m, 1H), 6.18-6.10 (dd, 1H), 4.98-4.94 (m, 1H), 4.62-4.61(m, 1H), 4.17-3.97(m, 1H), 3.82-3.74(m, 1H), 3.51-3.46(m, 1H), 2.94-2.35(m, 8H), 2.19-2.15(m, 1H), 1.71-1.51(m, 7H)

### EXAMPLE 4

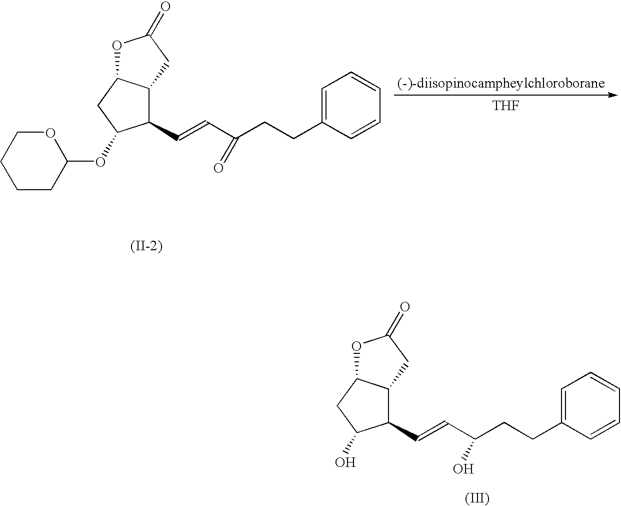

0.7 g of the compound of the formula (II-2) and 7 ml of THF were added into a flask. Under −60° C.˜−75° C., 3.5 g of (−)-chlorodiisopinocamphenylborane solution (62.5% in heptane) was added into the mixture dropwise. The mixture was stirred for 26 hours, and warm to room temperature. After the reaction was completed, the steps for completing the reaction was the same as illustrated in Example 2. Finally, the compound of the formula (III) was obtained, and the de value of which is 93%.

### EXAMPLE 5

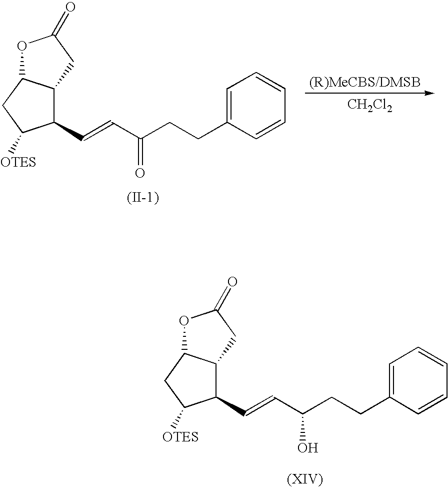

2.26 g of the compound of the formula (II-1), and 22.6 ml of CH₂Cl₂ were added into a 100 ml three-necked flask. Under −20° C. 0.65 ml of (R)-MeCBS)(1M) and 1.77 ml of DMSB (2M in THF) were added into the mixture, and the mixture was stirred for 5 hours. After the reaction was completed, 22.6 ml of methanol was added thereto to stop the reaction. The solvent in the mixture was removed by concentration under reduced pressure, and then 100 ml ethyl acetate and 50 ml water were used for extracting the mixture. After the layers were formed, the upper later was separated. Sodium sulfate was added into the upper layer for dehydration. After sodium sulfate was filtered out, the filtrate was concentrated under reduced pressure to get 2.32 g of a yellow oil-like product (XIV), and the de value of which is 70%.

### EXAMPLE 6

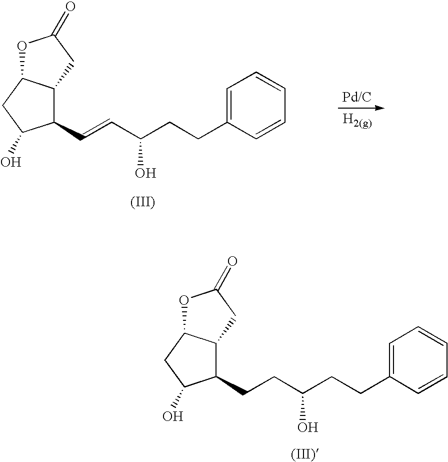

0.5 g of the compound of the formula (III), 0.05 g of Pd/C, and 10 ml of methanol were added into a 25 ml three-necked flask, and then the three-necked flask was filled with hydrogen under 30° C. After filling the hydrogen, the mixture was stirred for 1 hour, and HPLC was used to ensure the completion of the reaction. When the reaction was completed, the solid in the mixture was filtered out by celite. The filtrate was concentrated under reduced pressure to obtain an oil-like product. After analysis of the oil-like product by HPLC, 0.4 g of the compound of the formula (III)′ was obtained, and the yield of which is 83%.

### EXAMPLE 7

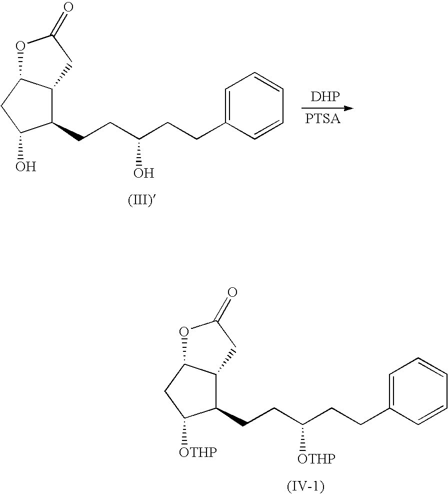

0.7 g of the compound of the formula (III)′, 15 ml of dichloromethane, and 0.043 g of PTSA were added into a 50 ml three-necked flask. Under the nitrogen-filled condition, DHP/dichloromethane solution (0.423 g of DHP in 5 ml dichloromethane solution) was added thereto at 20-25° C. After the mixture was stirred for 2.5 hours, TLC was used to ensure the completion of the reaction. When the reaction was completed, 5 ml of NaHCO_{3(aq)} was added into the mixture, and then the mixture was extracted by 25 ml ethyl acetate. After the layers were formed, the upper layer was separated, and 5 g of sodium sulfate was added into the upper layer for dehydration. After sodium sulfate was filtered out, the filtrate was concentrated under reduced pressure to obtain 1.73 g of a yellow oil-like product. Finally, the yellow oil-like product was purified by column chromatography, and 0.9 g of a light-yellow oil-like product (IV-1) was obtained.

¹H NMR(CDCl₃):

δ: 7.35-7.12(m,5H), 5.01-4.92(q,1H), 4.70-4.57(m,2H), 3.98-3.74(m,3H), 3.73-3.36(q,1H), 3.34-3.2(m,2H), 3.83-2.40(m,5H), 2.21-2.13(d,1H), 1.90-1.40(m,20H)

### EXAMPLE 8

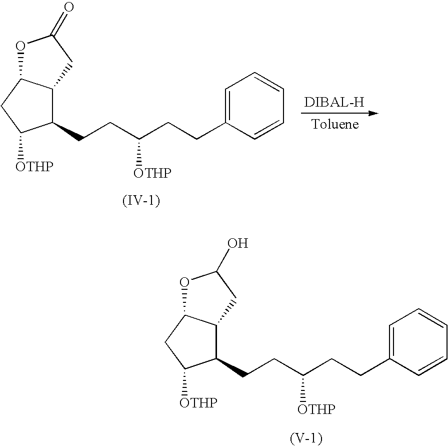

0.9 g of the compound of the formula (IV-1), and 15 ml of toluene were added into a 50 ml three-necked flask. Under the nitrogen-filled condition, the mixture was cooled down to −60˜−70° C., and then 1.42 g of DIBAL-H (1M, density=0.7 g/ml) was added thereto. The mixture was stirred for 30 min, and TLC was used to ensure the completion of the reaction. When the reaction was completed, the dry-ice bath was removed, and then 15 ml of NaHCO_{3(aq)} was added into the mixture. After the mixture was stirred for 30 min, the mixture was filtered by celite. Then, 10 ml of water was added into the filtrate to extract it. After the layers were formed, the upper layer was separated, and sodium sulfate was added into the upper layer for dehydration. After sodium sulfate was filtered out, the filtrate was concentrated under reduced pressure to obtain 0.9 g of a light-yellow oil-like product (V-1).

¹H NMR(CDCl₃):

δ: 7.31-7.09(m,5H), 5.7-5.4(1H), 4.75-4.57(m,3H), 3.98-3.60(m,4H), 3.56-3.4(m,2H), 2.90-2.59(m,2H), 2.40-2.20(m,3H), 2.19-2.0(m,2H), 1.95-1.40(m,17H), 1.40-1.20(m,3H)

### EXAMPLE 9

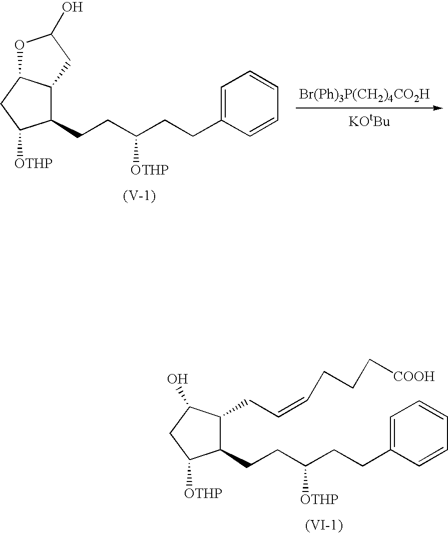

1.93 g of 4-carboxybutyl triphenylphosphonium bromide and 15 ml of THF were added into a 50 ml three-necked flask. After the mixture was cooled down to 0-10° C., 1.46 g of Potassium tetra-butoxide (KO^{t}Bu) was added thereto to form a reddish orange ylide. After the mixture was stirred for 30 min, the compound of formula (V-1)/THF solution (0.9 g of the compound of the formula (V-1) in 5 ml THF) was added thereto. After reaction for 1.0 hour, 10 ml of NaHCO_{3(aq)} and 20 ml of ethyl acetate were used for extraction of the mixture. After the layers were formed, the upper layer was separated, and sodium sulfate was added into the upper layer for dehydration. After sodium sulfate was filtered our, the filtrate was concentrated under reduced pressure to obtain a light-yellow oil-like product (VI-1). Sequentially, the light-yellow oil-like product (VI-1) was used for the next process directly.

### EXAMPLE 10

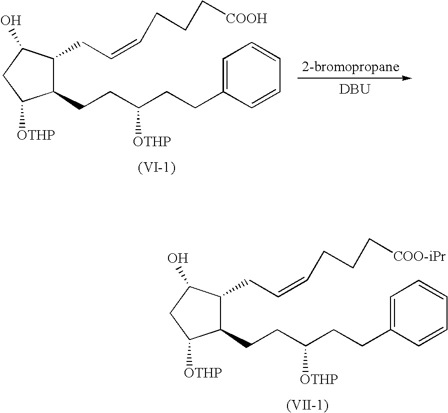

39 g of the compound of the formula (VI-1) was dissolved in 240 ml of acetone, and then 5.7 g of DBU was added thereto under 20-25° C. After the mixture was stirred for 10 min, 45 g of 2-bromopropane was added into the mixture. After the mixture was stirred for 12 hours, TLC was used to ensure the completion of the reaction. When the reaction was completed, 32% of HCl was used to adjust the pH of the mixture to 2.0˜6.0. Then, 100 ml of water and 500 ml of ethyl acetate was added into the mixture for extraction. After the layers were formed, the upper layer was separated and concentrated under reduced pressure to get 50 g of a yellow oil-like product (VII-1).

### EXAMPLE 11

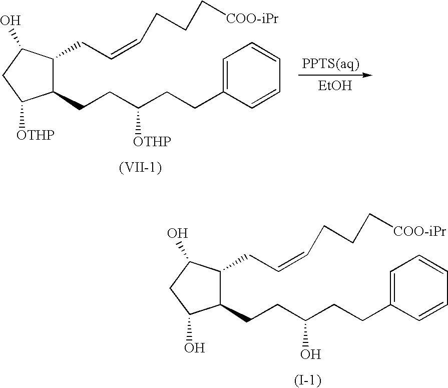

50 g of the compound of the formula (VII-1) was dissolved in 250 ml of ethanol, and 5 g of PPTS was added thereto under 20-25° C. Then, the mixture was heated to 50° C., and stirred for 3 hours. TLC was used to ensure the completion of the reaction. When the reaction was completed, 500 ml of ethyl acetate was used for substitution of ethanol, and then 100 ml of water and 200 ml of ethyl acetate was used for extraction. After the layers were formed, the upper layer was separated, and 40 g of sodium sulfate was added into the upper layer for dehydration. After sodium sulfate was filtered out, the filtrate was concentrated under reduced pressure to obtain 23.5 g of a yellow oil-like product. Finally, the yellow oil-like product was purified by column chromatography to obtain 13.5 g of a light-yellow oil-like product (I-1).

Rf=0.35(column: silica gel, mobile phase: ethyl acetate/hexane=7/3)

[α]_{D}²⁰=+31.82 (C(concentration)=0.9, acetonitrile)

¹H NMR(CDCl₃):

δ: 7.26-7.13(m,5H), 5.46-5.34(m,2H), 4.99-4.94(m,1H), 4.12-4.08(m,1H), 3.92(m, 1H), 3.63-3.61(m,1H), 2.79-2.77(m,1H), 2.65-2.62(m,1H), 2.26-2.23(m,4H), 2.10-2.07(m,2H), 1.83(m,2H), 1.75-1.73(m,2H), 1.67-1.33(m,8H), 1.24-1.18(d,6H)

¹³C NMR(CDCl₃):

δ: 173.51, 142.09, 129.49, 129.34, 128.36(4C), 125.76, 78.67, 74.55, 71.26, 67.64, 52.71, 51.79, 42.46, 38.99, 35.74, 34.03, 32.08, 29.64, 26.82, 26.58, 24.89, 21.79(2C)

MS: m/z=455(M+Na)

### EXAMPLE 12

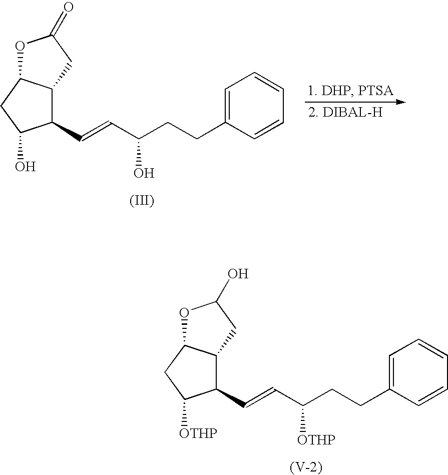

5.53 g of the compound of the formula (III), 60 ml of THF, and 0.1 g of PTSA were added into a 250 ml three-necked flask. In the nitrogen-filled condition, 5 ml of DHP was added into the mixture at 20-25° C. After the mixture was stirred for 5.5 hours, TLC was used to ensure the completion of the reaction. When the reaction was completed, the mixture was cooled down to −60˜−70° C., and 25 ml of 1M DIBAL-H was added thereto. The mixture was stirred for 15 hours, and TLC was used to ensure the completion of the reaction. When the reaction was completed, the dry-ice bath was removed, 1.5 ml of water and 55 ml of saturated sodium sulfate solution (i.e. water, sodium bicarbonate, and sodium sulfate) were added into the mixture, and the mixture was stirred. Then, 7 g of celite was added into the flask, and the mixture was stirred for 20 min, so that the mixture was filtered by celite. 15 ml of saturated sodium sulfate solution was added into the filtrate to extract it. After the layers were formed, the upper layer was separated, and sodium sulfate was added thereto for dehydration. Finally, after filtering out the sodium sulfate, the filtrate was concentrated under reduced pressure to obtain 8.8g of a yellow oil-like product (V-2). The recovery rate of the yellow oil-like product (V-2) was 102.2%.

### EXAMPLE 13

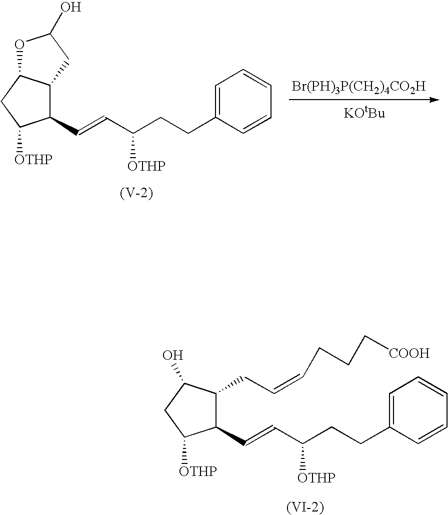

20.64 g of 4-carboxybutyl triphenylphosphonium bromide and 60 ml of THF were added into a 250 ml three-necked flask. After the mixture was cooled down to 0˜10° C., 15.64 g of KO^{t}Bu was added thereto to form a reddish orange ylide. After the mixture was stirred for 40 min, the mixture was cooled down to −10˜−5° C. Then, the compound of the formula (V-2)/THF solution (8.8 g of the compound of the formula (V-2) in 15 ml of THF) was added into the mixture. After reaction for 2 hours, 90 ml of water and 70 ml of Methyl tert-butyl ether (MTBE) were added into the mixture to extract it. After the layers were formed, the lower layer was separated, and 16% HCl_{(aq)} was used to adjust the pH of the lower layer to 6˜6.5. Then, 90 ml of MTBE was used three times for extraction. Finally, sodium sulfate was added into the organic layer for dehydration. After sodium sulfate was filtered out, the filtrate was concentrated under reduced pressure to obtain 16.8 g of an oil-like product (VI-2). Sequentially, the oil-like product (VI-2) was used for the next process directly.

### EXAMPLE 14

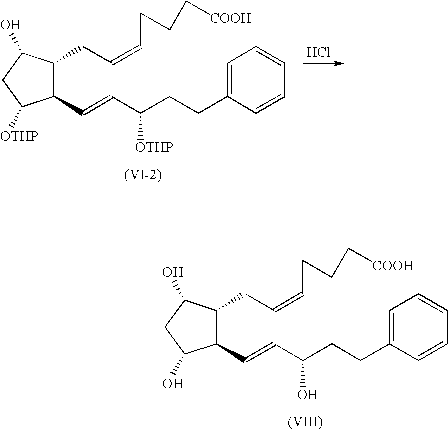

16.8 g of the compound of the formula (VI-2), 90 ml of acetone, and 90 ml of water were added into a 250 ml three-necked flask, and the pH of the mixture was adjusted to 0.7 by 16% HCl_{(aq)}. Then, the reaction was performed for 2 hours. After the mixture was concentrated under reduced pressure, MTBE and 100 ml of 1N NaOH_{(aq)} were added thereto, and the mixture was stirred for 15 min. After the layers were formed, the lower layer was separated, and 75 ml of MTBE was added thereto to extract the mixture six times. Then, the pH of the mixture was adjusted to 3˜4 by 16% HCl_{(aq)}. Again, the mixture was extracted by using 75 ml of MTBE four times. The upper layer was separated, and sodium sulfate was added thereto for dehydration. Finally, sodium sulfate was filtered out, and the filtrate was concentrated under reduced pressure to obtain 8.44 g of an oil-like product (VIII).

¹H NMR (CDCl₃):

δ: 7.16-7.28 (m, 5H), 5.35-5.59 (m, 4H), 4.56 (bs, 4H, OHx4),4.11-4.16 (m, 2H), 3.9 (m, 1H), 2.63-2.68 (m, 2H), 1.62-2.34 (m, 14H).

### EXAMPLE 15

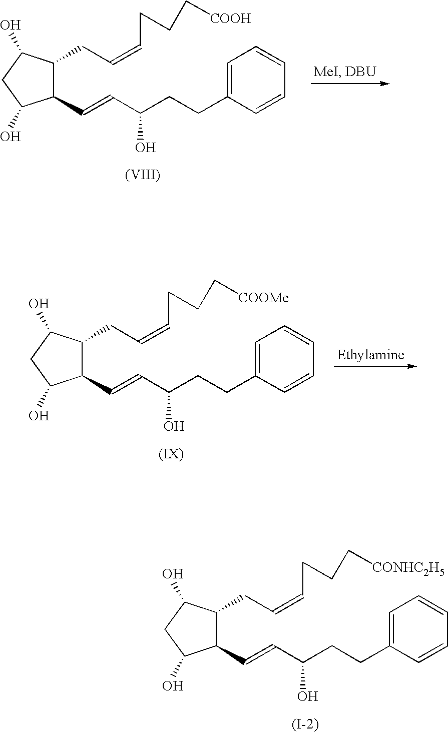

8.44 g of the compound of the formula (VIII) and 85 ml of acetone were added into a 250 ml three-necked flask. Under 20-35° C., 6.6 ml of DBU was added into the mixture. After the mixture was stirred for 35 min, 5.4 ml of MeI was added thereto. After the mixture was stirred for 3 hours, TLC was used for ensure the completion of the reaction. When the reaction was completed, the mixture was concentrated under reduced pressure, and then 120 ml of MTBE and 120 ml of water was added into the mixture to extract it. After the layers were formed, the lower layer was separated, and 100 ml of MTBE was added thereto three times for extraction. Then, the upper layer was collected, and 80 ml of water was added thereto four times for extraction. Finally, the upper layer was separated, and sodium sulfate was added into the upper layer for dehydration. After sodium sulfate was filtered out, the filtrate was concentrated under reduced pressure to obtain 7.85 g of an oil-like product (IX). Sequentially, the oil-like product (IX) was used for the next process directly.

7.85 g of the oil-like product (IX) and 125 ml of ethylamine aqueous solution (70%) were added into a 250 ml flask. After the mixture was stirred for 3 days, HPLC was used to ensure the completion of the reaction. When the reaction was completed, the mixture was concentrated under reduced pressure. Then, water and 140 ml of 0.75M sodium bisulfate were added into the mixture, and the pH of the mixture was adjusted to 7˜8.5. For extraction, 100 ml of ethyl acetate was added into the mixture four times, and then sodium sulfate was added into the organic layer for dehydration. After sodium sulfate was filtered out, the filtrate was concentrated under reduced pressure to obtain 7.13 g of an oil-like product. Finally, the oil-like product was applied into column chromatography, and eluted by ethyl acetate. After column chromatography, the product was crystallized in ethyl acetate to obtain 3.38g of the compound of the formula (I-2).

¹H NMR (CDCl₃):

δ: 7.13-7.25 (m, 5H), 6.08 (s, 1H), 5.29-5.59 (m, 4H), 4.03-4.10 (m, 2H), 3.87-3.90 (m, 1H), 3.50 (bs, 3H, OHx3), 3.18-3.23 (m, 2H), 2.60-2.70 (m, 2H), 1.20-2.38 (m, 14H), 1.07 (t, J=7.25 Hz, 3H)

¹³C NMR (CDCl₃):

δ: 14.7, 25.3, 25.6, 26.6, 31.8, 34.4,35.6, 38.7, 42.9, 50.2, 55.4, 72.2, 72.3, 77.7, 125.7, 128.3, 128.4, 129.1, 129.6, 133.1, 135.0, 142.0, 173.5

Although the present invention has been explained in relation to its preferred embodiment, it is to be understood that many other possible modifications and variations can be made without departing from the scope of the invention as hereinafter claimed.

What is claimed is:

1. A method for preparing a compound of the following formula (I), 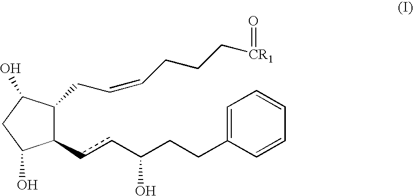
wherein
R₁ is C_{1˜6} alkoxy, or C_{1˜6} alkylamino, and [symbol] is a single bond or a double bound; which comprising:
(a) reacting a compound of the following formula (II) in the presence of (−)-chlorodiisopinocamphenylborane, 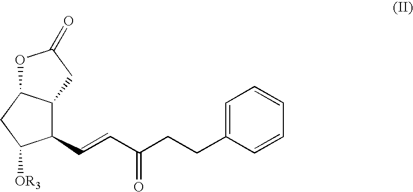
wherein
R₂ is tetrahydropyranyl substituted or unsubstituted with C_{1˜6} alkyl, or a protecting group as shown below: 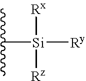
wherein R^{x}, R^{y}, or R^{z} are the same or different, and each independently C_{1˜6} alkyl, C_{6˜10} aryl, or C_{7˜16} arylalkyl,
and performing stereoselective reduction to form a compound of the following formula (III): 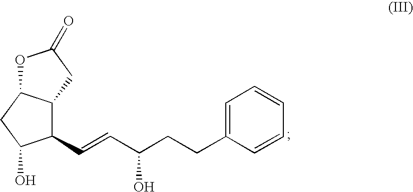
(b) performing protection reaction of the compound of formula (III) to form a compound of the following formula (IV), 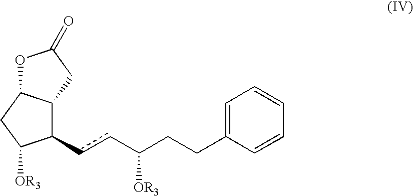
wherein
R₃ is tetrahydropyranyl substituted or unsubstituted with C_{1˜6} alkyl, or a protecting group as shown below: 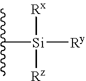
wherein R^{x}, R^{y}, or R^{z} are the same or different, and each independently C_{1˜6} alkyl, C_{6˜10} aryl, or C_{7˜16} arylalkyl;
(c) reducing the compound of the formula (IV) to form a compound of the following formula (V): 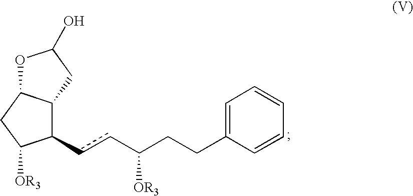
(d) performing Witting reaction of the compound of the following formula (V) with a compound represented as follow,
HOOC(CH₂)₄P⁺(R^{a})₃Y⁻
wherein R^{a} is C_{1˜6} alkyl, or C_{6˜10} aryl; Y is F, Cl, Br, or I,
to form a compound of the following formula (VI): 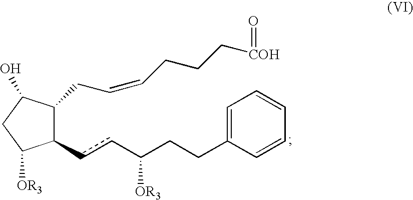
(e) performing esterification of the compound of the formula (VI) with a compound represented as follow,
R₄-Z
wherein R₄ is H, or C_{1˜6} alkyl, and Z is halogen, sulphate, mesyl, tosyl, or C_{1˜6} hydroxyl,
to form a compound of the following formula (VII): 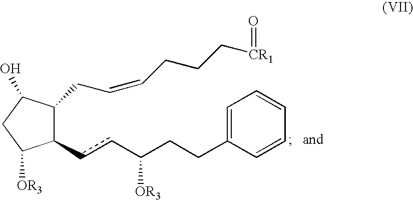
(f) deprotecting the compound of the formula (VII) to form a prostaglandin F analogue represented by the formula (I).

2. The method as claimed in claim 1, wherein the step (b) comprises step (b1) and step (b2):
(b1) hydrogenating the compound of the formula (III) to form a compound of the following formula (III)′: 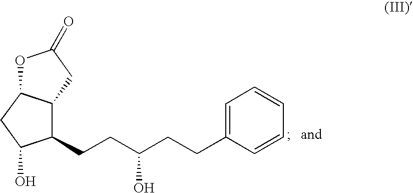
(b2) protecting the compound of the formula (III)′ to form the compound of the following formula (IV), 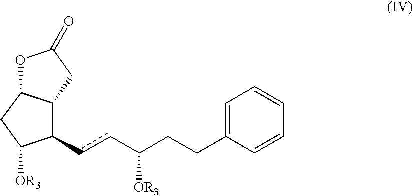
wherein
R₃ is tetrahydropyranyl substituted or unsubstituted with C_{1˜6} alkyl, or a protecting group as shown below: 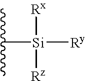
wherein R^{x}, R^{y}, or R^{z} are the same or different, and each independently C_{1˜6} alkyl, C_{6˜10} aryl, or C_{7˜16} arylalkyl.

3. The method as claimed in claim 1, wherein R₁ is isopropoxy, or ethylamino.

4. The method as claimed in claim 1, wherein R₂ is tetrahydropyranyl, triethylsilyl, tert-butyldimethylsilyl, t-butyldiphenylsilyl, or dimethylphenylsilyl.

5. The method as claimed in claim 1, wherein R₃ is tetrahydropyranyl, triethylsilyl, tert-butyldimethylsilyl, t-butyldiphenylsilyl, or dimethylphenylsilyl.

6. The method as claimed in claim 1, wherein the compound of the formula (I) is

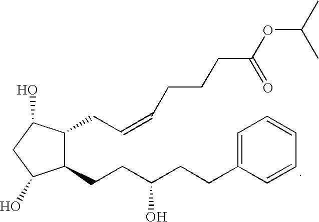

7. The method as claimed in claim 1, wherein the compound of the formula (I) is

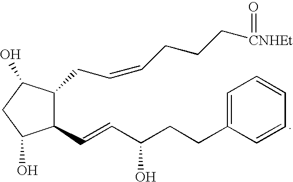

8. A method for preparing a compound of the following formula (I), 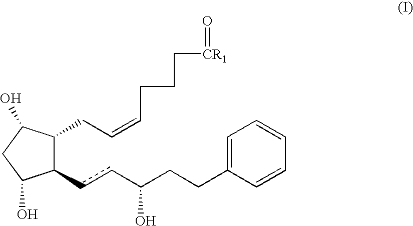
wherein
R₁ is C_{1˜6} alkoxy, or C_{1˜6} alkylamino, and [symbol] is a single bond or a double bound; which comprising:
(a) reacting a compound of the following formula (II) in the presence of (−)-chlorodiisopinocamphenylborane, 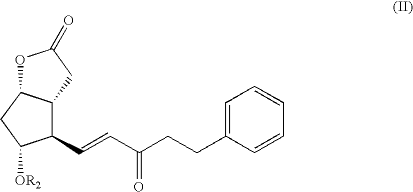
wherein
R₂ is tetrahydropyranyl substituted or unsubstituted with C_{1˜6} alkyl, or a protecting group as shown below: 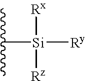
wherein R^{x}, R^{y}, or R^{z} are the same or different, and each independently C_{1˜6} alkyl, C_{6˜10} aryl, or C_{7˜16} arylalkyl,
and performing stereoselective reduction to form a compound of the following formula (III): 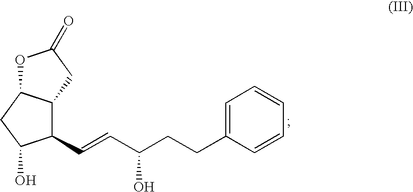
(b) performing protection reaction of the compound of formula (III) to form a compound of the following formula (IV), 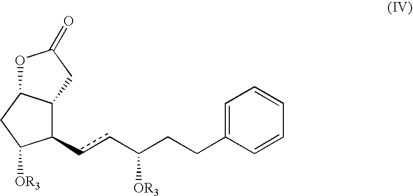
wherein
R₃ is tetrahydropyranyl substituted or unsubstituted with C_{1˜6} alkyl, or a protecting group as shown below: 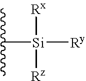
wherein R^{x}, R^{y}, or R^{z} are the same or different, and each independently C_{1˜6} alkyl, C_{6˜10} aryl, or C_{7˜16} arylalkyl;
(c) reducing the compound of the formula (IV) to form a compound of the following formula (V): 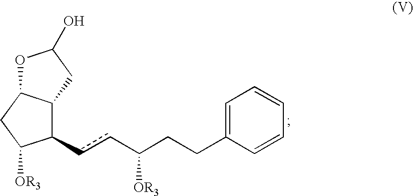
(d) performing Witting reaction of the compound of the following formula (V) with a compound represented as follow,
HOOC(CH₂)₄P⁺(R^{a})₃Y⁻
wherein R^{a} is C_{1˜6} alkyl, or C_{6˜10} aryl; Y is F, Cl, Br, or I,
to form a compound of the following formula (VI): 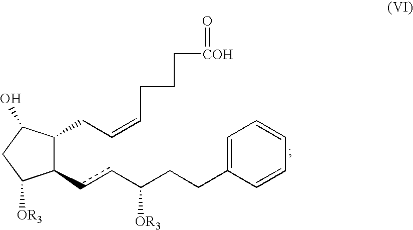
(e)′ deprotecting the compound of the formula (VI) to form a compound of the following formula (VIII): 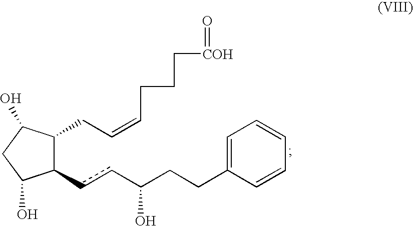
(f)′ performing esterification of the compound of the formula (VIII) to form a compound of the following formula (IX): 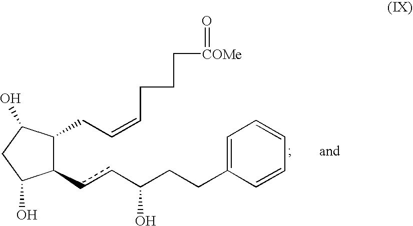
(g) performing amidation of the compound of the formula (IX) with a compound represented as follow,
R₅—NH₂
wherein R₅ is C_{1˜6} alkyl,
to form a prostaglandin F analogue represented by the formula (I).

9. The method as claimed in claim 8, wherein the step (b) comprises step (b1) and step (b2):
(b1) hydrogenating the compound of the formula (III) to form a compound of the following formula (III)′: 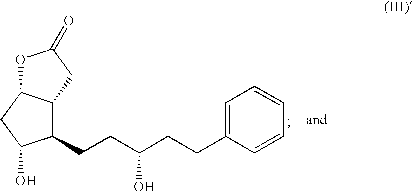
(b2) protecting the compound of the formula (III)′ to form the compound of the following formula (IV), 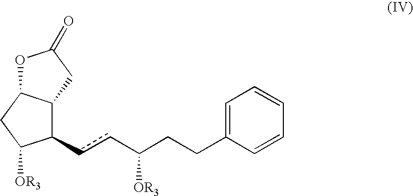
wherein
R₃ is tetrahydropyranyl substituted or unsubstituted with C_{1˜6} alkyl, or a protecting group as shown below: 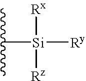
wherein R^{x}, R^{y}, or R^{z} are the same or different, and each independently C_{1˜6} alkyl, C_{6˜10} aryl, or C_{7·16} arylalkyl.

10. The method as claimed in claim 8, wherein R₁ is isopropoxy, or ethylamino.

11. The method as claimed in claim 8, wherein R₂ is tetrahydropyranyl, triethylsilyl, tert-butyldimethylsilyl, t-butyldiphenylsilyl, or dimethylphenylsilyl.

12. The method as claimed in claim 8, wherein R₃ is tetrahydropyranyl, triethylsilyl, tert-butyldimethylsilyl, t-butyldiphenylsilyl, or dimethylphenylsilyl.

13. The method as claimed in claim 8, wherein the compound of the formula (I) is

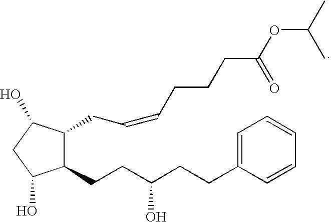

14. The method as claimed in claim 8, wherein the compound of the formula (I) is [chemical structure]
